(19) United States
(12) Patent Application Publication
(10) Pub. No.: US 2022/0080056 A1
(43) Pub. Date: Mar. 17, 2022

(54) DENDRIMER COMPOSITIONS AND USE IN TREATMENT OF NECROTIZING ENTEROCOLITIS AND OTHER GASTROINTESTINAL DISORDERS

(71) Applicant: The Johns Hopkins University, Baltimore, MD (US)

(72) Inventors: David Hackam, Baltimore, MD (US); Sujatha Kannan, Baltimore, MD (US); Kannan Rangaramanujam, Baltimore, MD (US); Diego F. Nino, Baltimore, MD (US); Fan Zhang, Baltimore, MD (US)

(21) Appl. No.: 17/484,175

(22) Filed: Sep. 24, 2021

Related U.S. Application Data
(63) Continuation of application No. 15/339,606, filed on Oct. 31, 2016, now abandoned.
(60) Provisional application No. 62/248,063, filed on Oct. 29, 2015.

(51) Int. Cl. A61K 49/00; A61K 31/19; A61K 9/48; A61K 31/198; A61K 9/20; A61K 9/107; A61K 9/10; A61K 47/59; A61K 9/00

(52) U.S. Cl. CPC A61K 49/0021; A61K 31/19; A61K 9/48; A61K 31/198; A61K 9/20; A61K 9/0053; A61K 9/10; A61K 49/0054; A61K 49/0032; A61K 47/595; A61K 9/107

(57) ABSTRACT

A dendrimer formulation, such as a PAMAM dendrimer or a multiarm PEG polymeric formulation has been developed for oral administration to the gastrointestinal tract for treatment of inflammatory diseases associated with infection or cancer. In the preferred embodiment, the dendrimers are in the form of dendrimer nanoparticles comprising poly(amidoamine) (PAMAM) hydroxyl-terminated dendrimers covalently linked to at least one therapeutic, prophylactic or diagnostic agent for treatment of one or more symptoms of necrotizing enterocolitis.

### CROSS-REFERENCE TO RELATED APPLICATIONS

This application claims priority to U.S. Ser. No. 62/248,063, filed on Oct. 29, 2015, which is incorporated herein in its entirety.

### STATEMENT REGARDING FEDERALLY SPONSORED RESEARCH OR DEVELOPMENT

This invention was made with Government Support under Agreement 1R01HD076901-01A1 (KR) and 1 R01 HD069562-01A1 (SK) awarded to Kannan Rangaramanujam and Sujatha Kannan, respectively, by the National Institutes of Health (NIH-NICHD). The Government has certain rights in the invention.

### FIELD OF THE INVENTION

This invention relates to oral formulations of poly(amidoamine) dendrimers for the treatment and/or diagnosis of inflammatory and/or infectious disorders such as necrotizing enterocolitis.

### BACKGROUND OF THE INVENTION

Necrotizing enterocolitis (NEC) is a severe inflammatory condition that affects the gastrointestinal tract of premature infants, and is characterized by the sudden development of intestinal necrosis followed by systemic sepsis and death in over 40% of cases. In those children who survive the onset of NEC, approximately 15% develop severe neurological injury, which is characterized by severely impaired cognition. Systemic inflammation, and neuroinflammation are the key known consequences associated with NEC.

Necrotizing enterocolitis is an acquired disease, primarily of preterm or sick neonates, characterized by mucosal or even deeper intestinal necrosis. It is the most common GI emergency among neonates. Symptoms and signs include feeding intolerance, lethargy, temperature instability, ileus, bloating, bilious emesis, hematochezia, reducing substances in the stool, apnea, and sometimes signs of sepsis. Diagnosis is clinical and is confirmed by imaging studies. Treatment is primarily supportive and includes nasogastric suction, parenteral fluids, TPN, and antibiotics. There currently exists no effective therapeutic or prophylactic approach for NEC and its associated systemic inflammation in premature infants. Treatment for a baby that may have necrotizing entercolis include halting regular feedings; relieving gas in the bowel by inserting a tube in the stomach; giving intravenous fluids and antibiotic medicines; monitoring the condition with abdominal x-rays, blood tests, and measurement of blood gases. The infant will need surgery if there is perforation of the intestines or inflammation of the abdominal wall (peritonitis). Surgery is used to remove dead bowel tissue, and may require a colostomy or ileostomy, and may require several weeks before the bowel can be reconnected.

It is therefore an object of the present invention to provide improved delivery to the gastrointestinal tract.

It is a further object of the invention to provide means of treating inflammatory and infectious disorders of the gastrointestinal tract, especially enterocolitis.

### SUMMARY OF THE INVENTION

A pharmaceutical composition including dendrimers delivering therapeutic, prophylactic and/or diagnostic agents can be administered orally to reach target cells in the gastrointestinal tract, for treatment of infection, inflammation, and cancer, as well as the brain. As demonstrated by the example, the formulation was effective in treating necrotizing enterocolitis (NEC), a severe inflammatory condition that affects the gastrointestinal tract of premature infants, and is characterized by the sudden development of intestinal necrosis followed by systemic sepsis and death in over 40% of cases. The development of NEC requires the activation of the bacterial receptor toll like receptor 4 (TLR4) on the intestinal epithelium, which leads to activation of the major immune cells of the brain, the microglia. Microglia activation initiates an inflammatory cascade which results in the loss of myelin in the prefrontal cortex, and the development of cognitive impairment in mice. Importantly, the structural and inflammatory changes that are observed in mice closely resemble the changes observed in humans who develop this disease, as revealed by immunostaining of sections of human brains obtained at autopsy.

Oral administration of poly(amidoamine) dendrimers target inflammation in the gastrointestinal (GI) tract as well as the central nervous system (CNS) and deliver drugs that are capable of producing functional improvements. Oral administration of the dendrimer leads to significant concentration of the dendrimer in the injured areas of the gut and the brain in mice with NEC, with further selective localization in the inflammatory cells. Strikingly, oral administration of an anti-inflammatory agent (N-acetyl cysteine) using dendrimer results in dramatic improvement of the brain injury and gut injury in animals with NEC. This selective localization of the dendrimer in the injured gut and brain demonstrates that the oral dendrimer formulation should be useful for non-surgical treatment of NEC with preservation of the gut along with treatment of the associated systemic inflammation including neuroinflammation resulting in brain injury. In addition, by selectively localizing the fluorophore-tagged dendrimer in the inflammatory cells in the gut and brain, this technology may also represent a diagnostic tool for sensitive detection of NEC.

In the preferred embodiment, the dendrimer is used for delivery to the gut and the brain of anti-inflammatory agents and TLR4 inhibitors conjugated to the dendrimer. This provides a non-surgical option of preserving the gut and prevention/treatment of associated brain injury in premature neonates with NEC, to prevent or alleviation injury of both the GI tract and the brain. By co-administering a diagnostic agent, the dendrimers can also be used to provide non-invasive, real time detection of inflammation and injury in the gut and brain in NEC. The selective localization of dendrimer nanodevices in cells associated with inflammation provides an approach for the non-invasive, real time detection of inflammation and injury in the gut and brain in NEC. A preferred diagnostic is a fluorophore approved for human use such as indocyanine green for non-invasive detection.

A preferred formulation includes PAMA dendrimer (4-6 generation) having N-acetyl cysteine bound thereto in combination with a TLR4 inhibitor. Preferred TLR4 inhibitors including small molecule inhibitors which inhibit TLR4 signaling, in particular C34 {Neal, 2013 PLoS One. 2013; 8(6): e65779.}.

### BRIEF DESCRIPTION OF THE FIGURES

FIGS. 1A, 1B and 1C demonstrate the significant decrease in microglial activation by Iba-1 staining in the periventricular region in NEC pups treated with D-NAC, by IHC and confirmed by quantification, that is similar to healthy controls (FIG. 1A). This is associated with increased glutathione levels and decreased protein nitration in pups with NEC that are treated with D-NAC (FIGS. 1B and 1C).

FIGS. 2A-2E demonstrate that D-NAC treatment prevents NEC induced cognitive injury: Pups (P21) with NEC that is untreated develop significant cognitive deficits at 3 weeks of age as seen by Y maze (FIG. 2A), novel object (FIG. 2B), Morris water maze (FIG. 2C) and morns water maze (FIG. 2D). These deficits are prevented upon treatment with D-NAC. D-NAC treatment leads to cognitive development that is similar to that of healthy controls. This is associated with improved myelination by IHC and upon quantification (FIG. 2E) when measured at P70.

### DETAILED DESCRIPTION OF THE INVENTION

### I. Definitions

The term “therapeutic agent” refers to an agent that can be administered to prevent or treat one or more symptoms of a disease or disorder. Examples include, but are not limited to, a nucleic acid, a nucleic acid analog, a small molecule, a peptidomimetic, a protein, peptide, carbohydrate or sugar, lipid, or surfactant, or a combination thereof.

The term “treating” refers to preventing or alleviating one or more symptoms of a disease, disorder or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The phrase “therapeutically effective amount” refers to an amount of the therapeutic agent that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

### II. Formulation

A. Dendrimers

The term “dendrimer” as used herein includes, but is not limited to, a molecular architecture with an interior core, interior layers (or “generations”) of repeating units regularly attached to this initiator core, and an exterior surface of terminal groups attached to the outermost generation. Examples of dendrimers include, but are not limited to, PAMAM, polyester, polylysine, and PPI. The PAMAM dendrimers can have carboxylic, amine and hydroxyl terminations and can be any generation of dendrimers including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. Dendrimers suitable for use with include, but are not limited to, polyamidoamine (PAMAM), polypropylamine (POPAM), polyethylenimine, polylysine, polyester, iptycene, aliphatic poly(ether), and/or aromatic polyether dendrimers. Each dendrimer of the dendrimer complex may be of similar or different chemical nature than the other dendrimers (e.g., the first dendrimer may include a PAMAM dendrimer, while the second dendrimer may comprise a POPAM dendrimer). In some embodiments, the first or second dendrimer may further include an additional agent. The multiarm PEG polymer includes a polyethylene glycol having at least two branches bearing sulfhydryl or thiopyridine terminal groups; however, embodiments disclosed herein are not limited to this class and PEG polymers bearing other terminal groups such as succinimidyl or maleimide terminations can be used. The PEG polymers in the molecular weight 10 kDa to 80 kDa can be used.

A dendrimer complex includes multiple dendrimers. For example, the dendrimer complex can include a third dendrimer; wherein the third-dendrimer is complexed with at least one other dendrimer. Further, a third agent can be complexed with the third dendrimer. In another embodiment, the first and second dendrimers are each complexed to a third dendrimer, wherein the first and second dendrimers are PAMAM dendrimers and the third dendrimer is a POPAM dendrimer. Additional dendrimers can be incorporated without departing from the spirit of the invention. When multiple dendrimers are utilized, multiple agents can also be incorporated. is not limited by the number of dendrimers complexed to one another.

As used herein, the term “PAMAM dendrimer” means poly(amidoamine) dendrimer, which may contain different cores, with amidoamine building blocks. The method for making them is known to those of skill in the art and generally, involves a two-step iterative reaction sequence that produces concentric shells (generations) of dendritic β-alanine units around a central initiator core. This PAMAM core-shell architecture grows linearly in diameter as a function of added shells (generations). Meanwhile, the surface groups amplify exponentially at each generation according to dendritic-branching mathematics. They are available in generations G0-10 with 5 different core types and 10 functional surface groups. The dendrimer-branched polymer may consist of polyamidoamine (PAMAM), polyester, polyether, polylysine, or polyethylene glycol (PEG), polypeptide dendrimers.

In accordance with some embodiments, the PAMAM dendrimers used can be generation 4 dendrimers, or more, with hydroxyl groups attached to their functional surface groups. The multiarm PEG polymer comprises polyethylene glycol having 2 and more branches bearing sulfhydryl or thiopyridine terminal groups; however, embodiments are not limited to this class and PEG polymers bearing other terminal groups such as succinimidyl or maleimide terminations can be used. The PEG polymers in the molecular weight 10 kDa to 80 kDa can be used.

In some embodiments, the dendrimers are in nanoparticle form and are described in detail in international patent publication No. WO2009/046446.

Preparation of PAMAM-NAC

Below is a synthetic scheme for conjugating N-acetylcysteine to an amine-terminated fourth generation PAMAM dendrimer (PAMAM-NH₂), using N-succinimidyl 3-(2-pyridyldithio)propionate (SPDP) as a linker.

Synthesis of N-succinimidyl 3-(2-pyridyldithio)propionate (SPDP) is performed by a two-step procedure, Scheme 1. First, 3-mercaptopropionic acid is reacted by thiol-disulfide exchange with 2,2′-dipyridyl disulfide to give 2-carboxyethyl 2-pyridyl disulfide. To facilitate linking of amine-terminated dendrimers to SPDP, the succinimide group is reacted with 2-carboxyethyl 2-pyridyl disulfide to obtain N-succinimidyl 3-(2-pyridyldithio)propionate, by esterification with N-hydroxysuccinimide by using N,N′-dicyclohexylcarbodiimide and 4-dimethylaminopyridine.

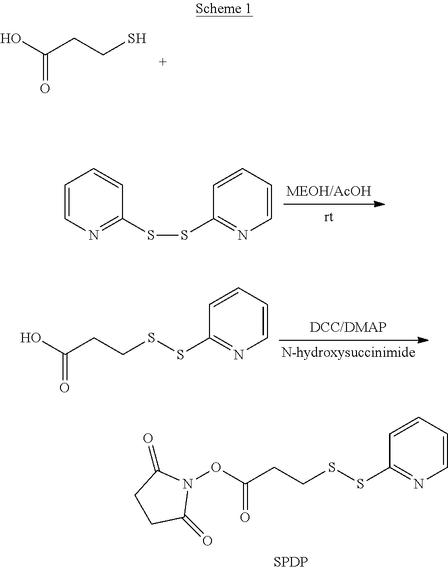

To introduce sulfhydryl-reactive groups, PAMAM-NH₂ dendrimers are reacted with the heterobifunctional cross-linker SPDP, Scheme 2. The N-succinimidyl activated ester of SPDP couples to the terminal primary amines to yield amide-linked 2-pyridyldithiopropanoyl (PDP) groups, Scheme 2. After the reaction with SPDP, PAMAM-NH-PDP can be analyzed using RP-HPLC to determine the extent to which SPDP has reacted with the dendrimers.

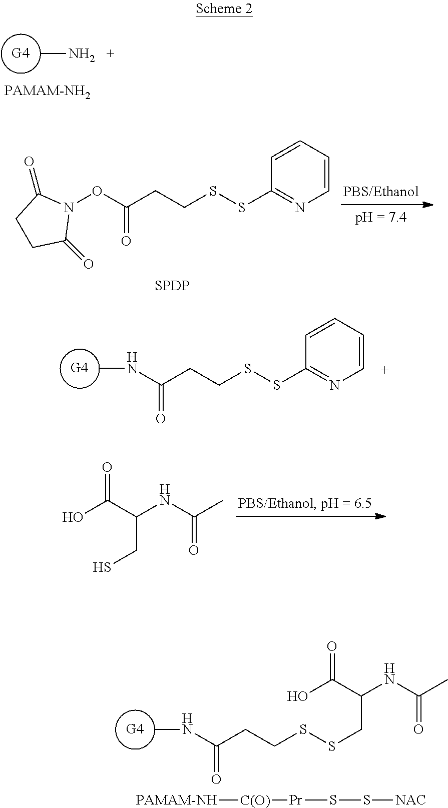

In another embodiment, the synthetic routes described in Scheme 4, below, can be used in order to synthesize D-NAC up to the pyridyldithio (PDP)-functionalized dendrimer 3. Compound 3 is then reacted with NAC in DMSO, overnight at room temperature to obtain D-NAC 5.

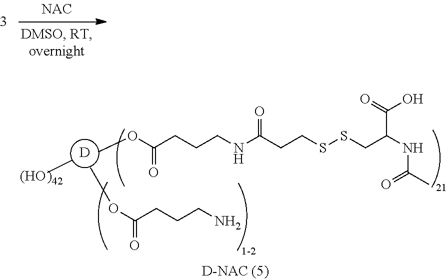

Preparation of Dendrimer-PEG-valproic acid conjugate (D-VPA)

Initially, valproic acid is functionalized with a thiol-reactive group. A short PEG-SH having three repeating units of (CH₂)₂O— is reacted with valproic acid using DCC as coupling reagent as shown in Scheme 3. The crude PEG-VPA obtained is purified by column chromatography and characterized by proton NMR. In the NMR spectrum, there was a down-shift of the peak of CH₂ protons neighboring to OH group of PEG to 4.25 ppm from 3.65 ppm that confirmed the formation of PEG-VPA. Although the thiol group also may be susceptible to reacting with acid functionality, the NMR spectra did not indicate any downward shift of the peak belonging to CH₂ protons adjacent to thiol group of PEG. This suggests that the thiol group is free to react with the thiol-reactive functionalized dendrimer.

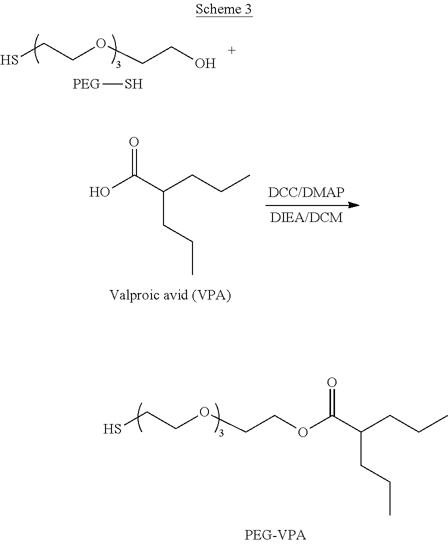

To conjugate PEG-VPA to the PAMAM-OH, a disulfide bond is introduced between the dendrimer and valproic acid, Scheme 4. First the dendrimer is converted to a bifunctional dendrimer 1 by reacting the dendrimer with fluorenylmethyloxycarbonyl (Fmoc) protected γ-aminobutyric acid (GABA). Conjugation of PEG-VPA to the bifunctional dendrimer involved a two-step process: the first step is the reaction of amine-functionalized bifunctional dendrimer 1 with N-succinimidyl-3-(2-pyridyldithio)-propionate (SPDP), and the second step involves conjugating the thiol-functionalized valproic acid. SPDP is reacted with the intermediate 2 in the presence of N,N-diisopropylethylamine (DIEA) to obtain pyridyldithio (PDP)-functionalized dendrimer 3.

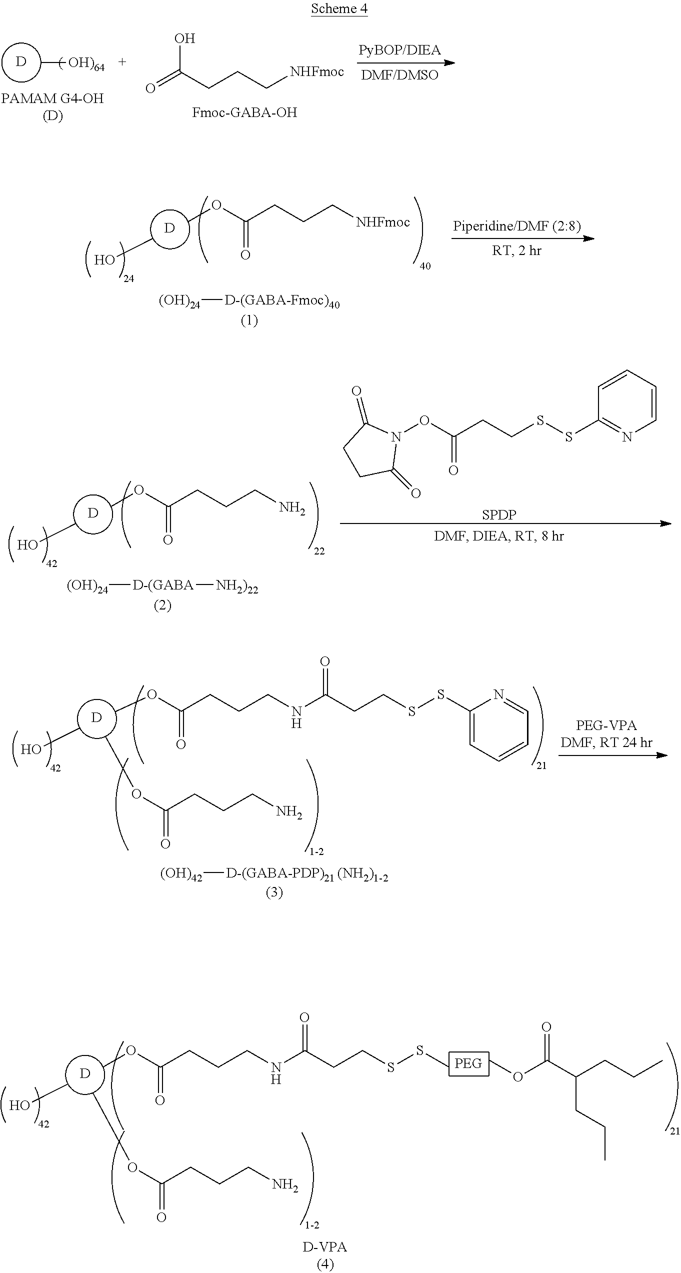

Even though this is an in situ reaction process, the structure was established by ¹H NMR. In the spectrum, new peaks between 6.7 and 7.6 ppm for aromatic protons of pyridyl groups confirmed the formation of the product. The number of pyridyl groups and number of GABA linkers were verified to be the same, which indicates that most of the amine groups reacted with the SPDP. Since this is a key step for the conjugation of the drug to the dendrimer, the use of mole equivalents of SPDP per amine group and time required for the reaction was validated. Finally, the PEG-VPA is reacted with the PDP-functionalized dendrimer in situ to get dendrimer-PEG-valproic acid (D-VPA). The formation of the final conjugate and loading of VPA were confirmed by ¹H NMR, and the purity of the conjugate was evaluated by reverse-phase HPLC. In the NMR spectrum, multiplets between 0.85 and 1.67 ppm for aliphatic protons of VPA, multiplets between 3.53 and 3.66 ppm for CH₂ protons of PEG, and absence of pyridyl aromatic protons confirmed the conjugate formation. The loading of the VPA is ˜21 molecules, estimated using a proton integration method, which suggests that 1-2 amine groups are left unreacted. In the HPLC chart, the elution time of D-VPA (17.2 min) is different from that for G4-OH (9.5 min), confirming that the conjugate is pure, with no measurable traces of VPA (23.4 min) and PEG-VPA (39.2 min).The percentage of VPA loading to the dendrimer is ˜12% w/w and validates the method for making gram quantities in three different batches.

B. Coupling Agents and Spacers

Dendrimer complexes can be formed of therapeutically active agents or compounds (hereinafter “agent”) conjugated or attached to a dendrimer or multiarm PEG. The attachment can occur via an appropriate spacer that provides a disulfide bridge between the agent and the dendrimer. The dendrimer complexes are capable of rapid release of the agent in vivo by thiol exchange reactions, under the reduced conditions found in body.

The term “spacers” as used herein is intended to include compositions used for linking a therapeutically active agent to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

The spacer can be chosen from among a class of compounds terminating in sulfhydryl, thiopyridine, succinimidyl, maleimide, vinylsulfone and carbonate group. The spacer can comprise thiopyridine terminated compounds such as dithiodipyridine, N-Succinimidyl 3-(2-pyridyldithio)-propionate (SPDP), Succinimidyl 6-(3[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio)toluene, (3-[2-pyridithio]propionyl hydrazide, The spacer can have maleimide terminations wherein the spacer comprises polymer or small chemical entity such as bis-maleimido diethylene glycol and bis-maleimido triethylene glycol, Bis-Maleimidoethane, bismaleimidohexane. The spacer can comprise vinylsulfone such as 1,6-Hexane-bis-vinylsulfone. The spacer can comprise thioglycosides such as thioglucose. The spacer can be reduced proteins such as bovine serum albumin and human serum albumin, any thiol terminated compound capable of forming disulfide bonds The spacer can include polyethylene glycol having maleimide, succinimidyl and thiol terminations.

C. Therapeutic, Prophylactic and Diagnostic Agents

The term “dendrimer complexes” as used herein refers to the combination of a dendrimer with a therapeutically active agent. These dendrimer complexes include an agent that is attached or conjugated to PAMAM dendrimers or multiarm PEG, which are capable of preferentially releasing the drug intracellularly under the reduced conditions found in vivo. The dendrimer complex, when administered by i.v. injection, can preferentially cross the blood brain barrier (BBB) only under diseased condition and not under normal conditions.

The therapeutically active agent, imaging agent, and/or targeting moiety can be either covalently attached or intra-molecularly dispersed or encapsulated. The dendrimer is preferably a PAMAM dendrimer up to generation 10, having carboxylic, hydroxyl, or amine terminations. The PEG polymer is a star shaped polymer having 2 or more arms and a molecular weight of 10 kDa to 80 kDa. The PEG polymer has sulfhydryl, thiopyridine, succinimidyl, or maleimide terminations. The dendrimer is linked to the targeting moiety, imaging agents, and/or therapeutic agents via a spacer ending in disulfide, ester or amide bonds.

Representative therapeutic (including prodrugs), prophylactic or diagnostic agents can be peptides, proteins, carbohydrates, nucleotides or oligonucleotides, small molecules, or combinations thereof. Examplary therapeutic agents include anti-inflammatory drugs, antiproliferatives, chemotherapeutics, vasodilators, and anti-infective agents.

Antibiotics include beta-lactams such as penicillin and ampicillin, cephalosporins such as cefuroxime, cefaclor, cephalexin, cephydroxil, cepfodoxime and proxetil, tetracycline antibiotics such as doxycycline and minocycline, macrolide antibiotics such as azithromycin, erythromycin, rapamycin and clarithromycin, fluoroquinolones such as ciprofloxacin, enrofloxacin, ofloxacin, gatifloxacin, levofloxacin and norfloxacin, tobramycin, colistin, or aztreonam as well as antibiotics which are known to possess anti-inflammatory activity, such as erythromycin, azithromycin, or clarithromycin. A preferred antiinflammatory is an antioxidant drug including N-acetylcysteine. Preferred NSAIDS include mefenamic acid, aspirin, Diflunisal, Salsalate, Ibuprofen, Naproxen, Fenoprofen, Ketoprofen, Deacketoprofen, Flurbiprofen, Oxaprozin, Loxoprofen, Indomethacin, Sulindac, Etodolac, Ketorolac, Diclofenac, Nabumetone, Piroxicam, Meloxicam, Tenoxicam, Droxicam, Lornoxicam, Isoxicam, Meclofenamic acid, Flufenamic acid, Tolfenamic acid, elecoxib, Rofecoxib, Valdecoxib, Parecoxib, Lumiracoxib, Etoricoxib, Firocoxib, Sulphonanilides, Nimesulide, Niflumic acid, and Licofelone.

Representative small molecules include steroids such as methyl prednisone, dexamethasone, non-steroidal anti-inflammatory agents, including COX-2 inhibitors, corticosteroid anti-inflammatory agents, gold compound anti-inflammatory agents, immunosuppressive, anti-inflammatory and anti-angiogenic agents, anti-excitotoxic agents such as valproic acid, D-aminophosphonovalerate, D-aminophosphonoheptanoate, inhibitors of glutamate formation/release, such as baclofen, NMDA receptor antagonists, salicylate anti-inflammatory agents, ranibizumab, anti-VEGF agents, including aflibercept, and rapamycin. Other anti-inflammatory drugs include nonsteroidal drug such as indomethacin, aspirin, acetaminophen, diclofenac sodium and ibuprofen. The corticosteroids can be fluocinolone acetonide and methylprednisolone. The peptide drug can be streptidokinase.

Many inflammatory diseases may be linked to pathologically elevated signaling via the receptor for lipopolysaccharide (LPS), toll-like receptor 4 (TLR4). There has thus been great interest in the discovery of TLR4 inhibitors as potential anti-inflammatory agents. Recently, the structure of TLR4 bound to the inhibitor E5564 was solved, enabling design and synthesis of new TLR4 inhibitors that target the E5564-binding domain. These are described in U.S. Pat. No. 8,889,101, the contents of which are incorporated by reference. As reported by Neal, et al., PLoS One. 2013; 8(6): e65779e, a similarity search algorithm used in conjunction with a limited screening approach of small molecule libraries identified compounds that bind to the E5564 site and inhibit TLR4. The lead compound, C34, is a 2-acetamidopyranoside (MW 389) with the formula C₁₇H₂₇NO₉, which inhibits TLR4 in enterocytes and macrophages in vitro, and reduces systemic inflammation in mouse models of endotoxemia and necrotizing enterocolitis. Molecular docking of C34 to the hydrophobic internal pocket of the TLR4 co-receptor MD-2 demonstrated a tight fit, embedding the pyran ring deep inside the pocket. Strikingly, C34 inhibited LPS signaling ex-vivo in human ileum that was resected from infants with necrotizing enterocolitis. These findings identify C34 and the β-anomeric cyclohexyl analog C35 as novel leads for small molecule TLR4 inhibitors that have potential therapeutic benefit for TLR4-mediated inflammatory diseases.

Wipf, et al., Tetrahedron Lett. 2015 56(23):3097-3100 (“Wipf”), incorporated by reference herein, describes analogues of C34. A copper(II)-mediated solvolysis of anomeric oxazolines and an acid-mediated conversion of β-glucosamine and β-galactosamine pentaacetates were used to generate analogs of C34 at the anomeric carbon and at C-4 of the pyranose ring. These compounds were evaluated for their influence on TLR4-mediated inflammatory signaling in cultured enterocytes and monocytes. Their efficacy was confirmed using a NF-kB-luciferase reporter mouse, thus establishing the first structure-activity relationship (SAR) study in this series and identifying the more efficacious isopropyl 2-acetamido-α-galactoside 17. These data show that C34, its analogues, or other TLR4 inhibitors can be conjugated to the dendrimers for use in the oral formulations.

The dendrimer complex can also be used to deliver anti-excitotoxic and D-anti-glutamate agents. Preferred candidates are: MK801, Memantine, Ketamine, 1-MT.

Representative oligonucleotides include siRNAs, microRNAs, DNA, and RNA. The therapeutic agent can be a PAMAM dendrimer with amine or hydroxyl terminations.

The dendrimer complexes linked to a bioactive compound or therapeutically active agent can be used to perform several functions including targeting, localization at a diseased site, releasing the drug, and imaging purposes. The dendrimer complexes can be tagged with or without targeting moieties such that a disulfide bond between the dendrimer and the agent or imaging agent is formed via a spacer or linker molecule.

D. Devices and Formulations

The dendrimers can be administered enterally. The carriers or diluents used herein may be solid carriers or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

### III. Methods of Treatment

A. Disorders or Diseases to be Treated

The formulations can be administered to treat disorders associated with infection, inflammation, or cancer, particular those having systemic inflammation that extends to the CNS. The innate immune receptor toll-like receptor 4 (TLR4) has been recognized as the receptor on hematopoietic and non-hematopoietic cells for bacterial endotoxin (lipopolysaccharide, “LPS”), as well as for a variety of endogenous molecules that are released during inflammatory or infectious disorders. A number of diseases have been attributed to exaggerated TLR4 signaling, including both infectious and non-infectious processes. These include necrotizing enterocolitis (NEC), abdominal sepsis, pneumonia, arthritis, pancreatitis and atherosclerosis. In a preferred embodiment, the disease to be treated is NEC.

The dendrimer complex composition, including a dendrimer linked to a therapeutic, prophylactic or diagnostic agent, can selectively target microglia and astrocytes, which play a key role in the pathogenesis of NEC. N-acetyl cysteine (“NAC”) has been extensively investigated and studied. It is also investigated for neuro-inflammation associated in maternal fetal infections. However, NAC suffers from low bioavailability due to high plasma protein binding. The dendrimer complex compositions overcome the plasma protein binding without affecting the activity of NAC. G4 PAMAM-NAC can be ten to a hundred times more efficacious in vivo than the free drug NAC by single i.v. administration. The free drug NAC exhibits very high plasma protein binding resulting in reduced bioavailability. One of the major advantages of this dendrimer complex is that it enhances the bioavailability by restricting the unwanted drug plasma protein interactions and selectively results in rapid release of the drug intracellularly to exhibit the desired therapeutic action.

The high payload of the drug NAC in the G4 PAMAM-NAC requires very small quantities (10 mg) of the carrier, PAMAM dendrimer, thereby reducing the amounts administered daily. A decreased quantity of agent limits the side effects associated with the agent. Since the bioavailability of the agent remains high, the positive effects of the agent are not lowered despite the administration of smaller quantities of agent. The dendrimer complexes including the dendrimer-drug conjugates, restricts its biodistribution to tissues and organ and preferentially deliver the drug at the target site thereby reducing the undesired side effects.

Dendrimer complexes effectively transport across the BBB, and are therefore useful for targeted drug delivery in neurological, neurodevelopmental, and neurodegenerative disorders and brain injury. G4-PAMAM-S--S-NAC conjugates specifically target activated microglial cells and astrocytes in neuroinflammatory disorders:

The therapeutic efficacy of G4-PAMAM-S--S-NAC dendrimer conjugate was evaluated after two days of animal treatment with lipopolysaccharide (LPS) to induce white matter injury and hypomyelination in the developing rabbit brain (an animal model of Cerebral Palsy). NAC selectively delivered from the G4-PAMAM-S--S-NAC dendrimer complexes strongly suppressed pro-inflammatory cytokines (TNF-.alpha., IL-6 mRNA), inflammatory signaling factors, including NF.kappa.B and nitrotyrosine, and enhanced GSH level. The G4-PAMAM-S--S-NAC was found to be ten to a hundred times more efficacious compared with free NAC. This supports a conclusion that the G4-PAMAM-S--S-NAC traversed across the BBB. The targeted delivery of NAC from dendrimer complex to actived microglial cells improved the motor deficits and attenuated recovery from the LPS-induced brain injury in a neonatal rabbit model of cerebral palsy.

A significant reduction in proinflammatory cytokines (TNF-.alpha., IL-6 mRNA) was observed on administration of G4-PAMAM-S--S-NAC dendrimer complexes. The kits treated with NAC and G4-PAMAM-S--S-NAC showed a decrease in fetal inflammation response with improvement of motor deficits when compared to the kits that were treated with saline. The kits that were treated with G4-PAMAM-S--S-NAC conjugates had less behavioral changes and lower microglial activation in the brain when compared to the kits that received NAC alone due to the sustained delivery of NAC from G4-PAMAM-S--S-NAC conjugate. The results indicated that G4-PAMAM-S--S-NAC conjugates have a greater effect than NAC alone since it is preferentially taken up by activated macrophages and microglial cells, reducing the inflammatory and oxidative and nitrosative effects.

Treatment with G4-PAMAM-S--S-NAC dendrimer complexes reduced white matter injury and microglia activation. A significant reduction in dose of NAC was observed when administered as G4-PAMAM-S--S-NAC to elicit the similar response as that observed for free NAC. Both free NAC at concentration 100 mg/kg and G4-PAMAM-S--S-NAC at concentration 10 mg/kg, 10 mg elicit identical responses, demonstrating that on conjugating to dendrimer a reduction in dose is achieved. G4-PAMAM-S--S-NAC at lower concentrations than free NAC shows significant protective effects against LPS-induced brain injuries, suppression of TNF-.alpha. and down-regulation of IL-6 activity. This activity of the dendrimer-NAC conjugates may be attributed to its ability to interfere with the early inflammatory responses by blocking or modifying the signal transduction factor NF-.kappa.B and nitrotyrosine, thereby modulating cellular activation. 6 and 8 arm PEG-NAC conjugates released 74% of NAC in the intracellular GSH concentration (2 and 10 mM), within 2 hours. At a concentration range of between 0.008-0.8 mM, the conjugates were nontoxic to the microglial cells. At an equimolar concentration of NAC (0.5 mM) the 6-arm-PEG-S--S-NAC and 8-arm-PEG-S--S-NAC were more efficient in inhibition of GSH depletion than the free NAC. Both 6 and 8-arm-PEG-S--S-NAC conjugates, each at 0.5 mM and 5 Mm concentration showed significant inhibition in ROS production when compared to free NAC at equimolar concentrations. The studies demonstrate that the conjugates are superior in inhibition of the NO production as compared to the free NAC. At the highest concentration (5 mM), the free drug reduced the H₂O₂ levels and nitrite levels by 30-40%, whereas the conjugates reduced the H₂O₂ and nitrite levels by more than 70%. This shows that the conjugates are able to traffic the drug inside the cells, and release the drug in the free form and are significantly more efficacious than the free drug. At 5 mM concentration 6-arm-PEG-S--S-NAC conjugate (1) showed significant inhibition (70%) of TNF-.alpha. production when compared to equivalent concentration of NAC (Pb0.05). 8-arm-PEG-S--S-NAC conjugate (3) showed significant inhibition of TNF-.alpha. production (70%) at 5 mM when compared to equivalent concentration of NAC (Pb0.05 and Pb0.01). PEGylated NAC is a dendrimer complex with utility for the pharmaceutical industry, as PEGs are approved for human use and this device addresses limitations of NAC and provides greater efficacy.

B. Dosages

Typically, an attending physician will decide the dosage of the composition with which to treat each individual subject, taking into consideration a variety of factors, such as age, body weight, general health, diet, sex, compound to be administered, route of administration, and the severity of the condition being treated. The dose of the compositions can be about 0.0001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight

In general the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, monthly or yearly dosing.

It will be understood by those of ordinary skill that a dosing regimen used in the inventive methods can be any length of time sufficient to treat the disorder in the subject. The term “chronic” as used herein, means that the length of time of the dosage regimen can be hours, days, weeks, months, or possibly years.

The dendrimer complexes can be administered in combination with one or more additional therapeutically active agents, which are known to be capable of treating conditions or diseases discussed above.

The present invention will be further understood by reference to the following non-limiting examples.

### EXAMPLE 1

### Treatment of Necrotizing Enterocolitis

The development of NEC requires the activation of the bacterial receptor toll like receptor 4 (TLR4) on the intestinal epithelium, as mice lacking TLR4 are protected from NEC, while humans with NEC exhibit increased TLR4 activation in the gut. Activation of TLR4 on the intestinal epithelium leads to activation of microglia, resulting in the loss of myelin in the prefrontal cortex, and the development of cognitive impairment in mice in a manner that closely resembles the disease observed in humans.

Poly(amidoamine) dendrimers target inflammation in the CNS and deliver drugs to produce functional improvements. As demonstrated by the following example, oral administration of the dendrimer leads to significant accumulation of the dendrimer in the injured areas of the gut and the brain in mice with NEC, with further selective localization in the inflammatory cells. Strikingly, oral administration of an anti-inflammatory agent (N-acetyl cysteine) using dendrimer results in dramatic improvement of the brain injury and gut injury in animals with NEC.

Experimental NEC in rodents leads to a cognitive impairment that resembles the human NEC-associated brain injury, and required microglial-induced ROS release and demyelination. Remarkably, these effects can be prevented by oral administration of a dendrimer-NAC conjugate. This selective localization of the dendrimer in the injured gut and brain has implications for non-surgical treatment of NEC with preservation of the gut along with treatment of the associated systemic inflammation including neuroinflammation resulting in brain injury. The selective localization of the fluorophore-tagged dendrimer in the inflammatory cells in the gut can also be used as a diagnostic tool for sensitive detection of NEC.

Materials and Methods

Dendrimers. Detailed materials and methods used in the experiments below, including protocols for making the dendrimers-Cy5 and dendrimers-drug conjugates, have been described by Kannan S et al Sci. Transl. Med., 4:130ra46 (2012) and in U.S. Pat. No. 8,889,101.

Conjugation of dendrimer conjugates. The conjugation of dendrimers to Cy5 was done using previously reported methods (Kannan et al., Science Trans. Med (April, 2012). For drug experiments, dendrimers were conjugated to N-acetyl-cysteine and administered at doses ranging from 2-20 mg/kg at differing time points.

Statistical analysis. The data was analyzed for the reproducibility using Student's t-test to determine the significance between two groups. A p-value equal to or less than 0.05 was considered significant.

Animal model of NEC. Neonatal mice (postnatal day 6-11) were exposed to a well-established model of necrotizing enterocolitis (NEC). Briefly, pups were fed formula supplemented with bacteria isolated from human NEC via gavage five times/day and submitted to hypoxia (5% O₂, 95% N₂) for 10 min in a hypoxic chamber twice daily for 4 days.

Two experimental protocols were employed to determine the acute versus long-term effects of NEC on the brain. To determine acute effects, brain samples were harvested immediately after the completion of the NEC protocol (postnatal day 11) followed by immunohistochemical evaluation of myelination and microglial activation. Behavioral implications of NEC exposure were determined 3 weeks after the completion of NEC protocol (postnatal day 32-46) using a Morris water maze and novel object recognition tests.

Dendrimer-NAC therapies. N-acetyl-cysteine conjugated dendrimers (D-NAC) delivered through oral gavage at a clinically relevant dose of 18 mg/kg on a NAC basis (100 mg/kg on a D-NAC conjugate basis), on days 2 and 3 of the NEC protocol (i.e. postnatal days 8 and 9).

For imaging, the Cy5-labeled dendrimers (D-Cy5) were delivered through oral gavage at 100 mg/kg on day 3 of NEC protocol, with few hours delay after the last dose of D-NAC.

High Performance Liquid Chromatography (HPLC) analysis. The purity of the dendrimer-Cy5 conjugates (D-Cy5) were analyzed using a Waters HPLC instrument (Waters Corporation, Milford, Massachusetts) equipped with Waters In-line degasser, binary pump, photodiode array (PDA) detector, multi fluorescence λ detector and auto sampler (maintained at 4° C.) interfaced with Empower software. The HPLC chromatogram was monitored simultaneously for absorbance at 210 nm for dendrimer and 650nm for Cy5 using Waters 2998 PDA detector and fluorescence with excitation at 645 nm and emission at 662 nm using Waters 2475 fluorescence detector. The water/acetonitrile (0.1% w/w TFA) was freshly prepared, filtered, degassed, and used as a mobile phase. TSK-Gel ODS-80 Ts (250×4.6 mm, 25 cm length with 5 μm particle size) connected to TSK-Gel guard column was used. A gradient flow was used with initial condition being 90:10 (H20/ACN) and then gradually increasing the acetonitrile concentration to 10:90 (H20/ACN) in 30 min and returning to original initial condition 90:10 (H20/ACN) in 60 min with flow rate of 1 ml/min.

Immunohistochemistry and confocal microscopy. Brian slices were fixed in 2% paraformaldehyde (PFA) in PBS. The brains were frozen in 20% sucrose with optimum cutting temperature compound (OCT) (Sakura Finetek USA Inc., Torrance, Calif.) in a 1:2 ratio respectfully using dry ice in isopentane. Cryoblocks are stored at −80 ° C. until sectioned. Eight μm sections were cut from frozen blocks using a cryostat. Sections were incubated in rabbit anti-Ionised Calcium Binding Adapter 1 molecule (Iba-1) (Wako chemicals, USA), which is a microglia cell marker, and a goat anti-rabbit-Cy3 secondary antibody applied. Sections were analysed on a Zeiss 510 confocal microscope. Excitation and emission wavelengths and laser settings were identical to analyze all tissue in IV injected animals. Z-stacks of sections were taken and collapsed to give an image through the depth of the whole section.

Results

Pups exposed to NEC display a significant cognitive deficit, evidenced by impaired working memory and spatial learning in Morris water Maze and novel object recognition behavioral tests.

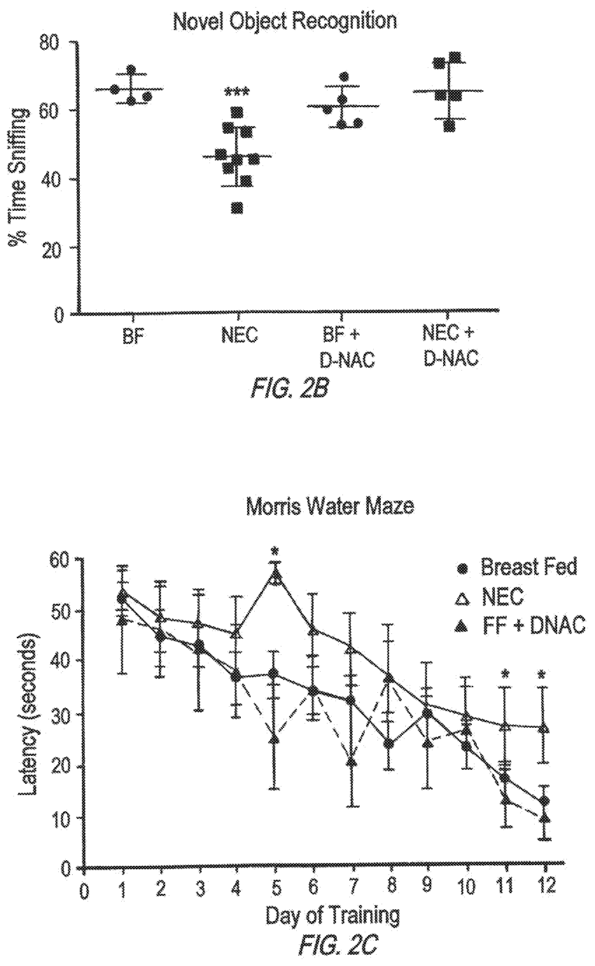

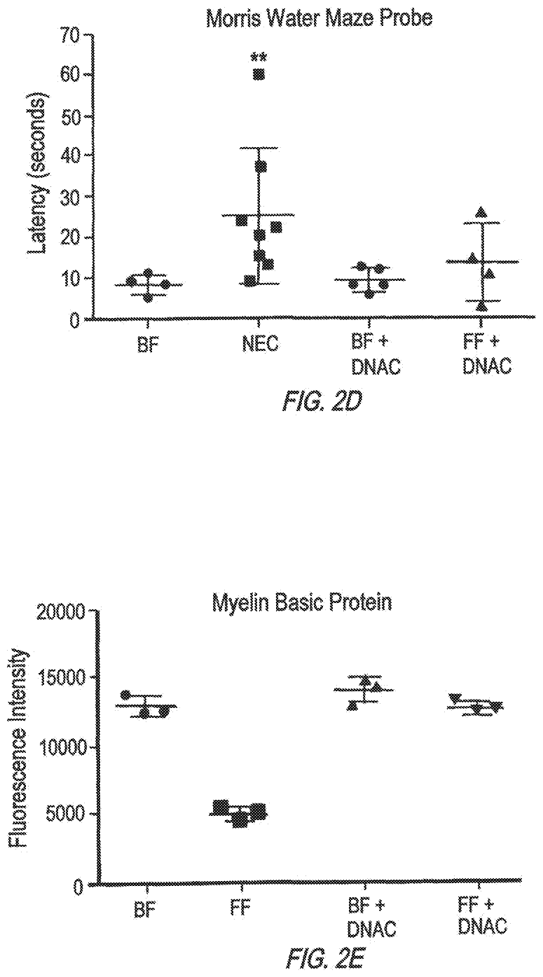

Behavioral assessments were performed in mice that reached weaning age (2-3 weeks after the induction of NEC). The mice that were submitted to experimental NEC were found to have significantly impaired cognitive function when compared to litter-mate breast-fed controls. This was manifested as a significant working memory deficit evidenced by the impaired performance in the Y Maze Spontaneous Alternation test (FIG. 2A). The percent spontaneous alternation performance was significantly reduced in the NEC animals suggesting impairment in active retrograde working memory. In addition, the performance of NEC animals in the novel object recognition test was significantly decreased (FIG. 2B), demonstrating impairment in the recognition memory and retention abilities of these mice compared to breast-fed controls. Furthermore, evaluation of spatial memory and learning in NEC animals using the Morris water maze test demonstrated a significant cognitive deficit, as evidenced by the increased latency to find the platform within the water maze 48 hours after the last training session (FIG. 2C). Furthermore, a deficient learning pattern was evident in the NEC animals when compared to breast-fed controls during the training period (FIG. 2D). Taken together, the findings demonstrate that experimental NEC leads to cognitive impairment that resembles the neurodevelopmental deficits observed in NEC survivors.

These findings are correlated with histopathological evaluations of brain myelination and microglial activation. Animals exposed to NEC have a deficient myelination pattern when compared to control animals, which is particularly evident by the decreased expression of myelin basic protein (MBP) in the midbrain and corpus callosum. Furthermore, NEC treated animals display increased microglial activation at the level of the corpus callosum, hippocampus and midbrain compared to controls. Decreased behavioral test performance was further correlated with white matter injury and a significant loss of myelin, as demonstrated by white matter depletion on cerebral MRI and electron microscopy.

Orally-administered dendrimers are taken up readily by the gut, and exhibited pathology-dependent biodistribution. The dendrimer was mostly seen to accumulate in the cerebral cortex, especially the parietal cortex and the motor cortex. D-Cy5 were also seen to accumulate at thalamus, thalamic nuclei.

Administration of D-NAC during NEC leads to a protective effect on the brain as evidenced by a reduced D-Cy5 uptake (indicative of a lower level of inflammation, reduced of microglial activation, and normal myelination pattern, compared to untreated NEC mice. The protective effects of D-NAC were observed despite the fact that animals display clear evidence of necrotizing enterocolitis by histopathological and qRT-PCR evaluation.

In all mice group dosed with Cy5-labeled dendrimers (D-Cy5), the dendrimers accumulated in the gut, taken up by the epithelial cells in the intestine villus, indicating D-Cy5 was absorbed in the GI track and partitioned in the systemic circulation.

In the formula fed (NEC) mice, Cy5 labeled dendrimers (D-Cy5) were mostly seen to accumulate in the cerebral cortex, especially the parietal cortex and the motor cortex (FIG. 2). D-Cy5 were also seen to accumulate at thalamus, thalamic nuclei.

To further identify the cellular co-localization of D-Cy5 at the region of interests, the somatosensory cortex at the parietal region was examined under higher magnification. At the somatosensory cortex, most of the D-Cy5 localization seem to be in the layer 2-5 of the cortex, and forms a pattern of cellular uptake.

At thalamus and thalamic nuclei, D-Cy5 clearly showed cellular co-localization, with dominate cell uptake by unlabeled cells, and some uptake by activated microglia/macrophages.

In addition, D-NAC treatment significantly decreased the NEC associated microglial activation, D-Cy5 accumulation in the periventricular region and greatly enhanced the myelination in the brains of formula fed mice. D-NAC treatment decreased the D-Cy5 accumulation in the periventricular region of the brain in the formula fed mice. In breast fed mice (healthy positive controls, D-Cy5 were only seen at the choroid plexus. In formula fed mice (NEC negative control, D-Cy5 accumulated at the periventricular region with a great amount and formed a scattered pattern, presumably due to cellular uptake. After D-NAC treatment to the formula fed mice (NEC dendrimer-NAC treated), the D-Cy5 accumulation decreased significantly around the periventricular region, with only minimal D-Cy5 observed in the outlayer of ventrical. Since the dendrimer uptake in brain has been shown to be proportional to the extent of inflammation, this indicates that oral D-NAC treatment reduced neuroinflammation.

D-NAC treatment decreased the NEC associated microglial activation. In breast fed and formula fed+D-NAC treatment mice, microglial cells had similar population, with small cell bodies, suggestion microglial cells were in ‘resting state’. In contrast, in the formula fed NEC mice (control), microglia population increased significantly, with enlarged cell bodies, suggesting microglial activation.

D-NAC treatment protected against the myelination defect associated with NEC in the brains of formula fed mice. The breast fed (healthy control) and formula fed+D-NAC treatment (NEC, treated with D-NAC) mice had a higher extent of myelination in the brain than the only formula fed mice (NEC untreated), indicating reduction of inflammation in the formula fed+D-NAC treatment group benefited the neurological repair

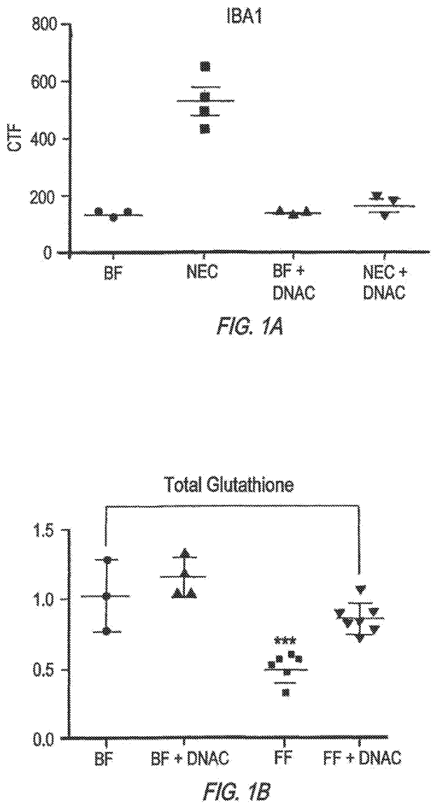

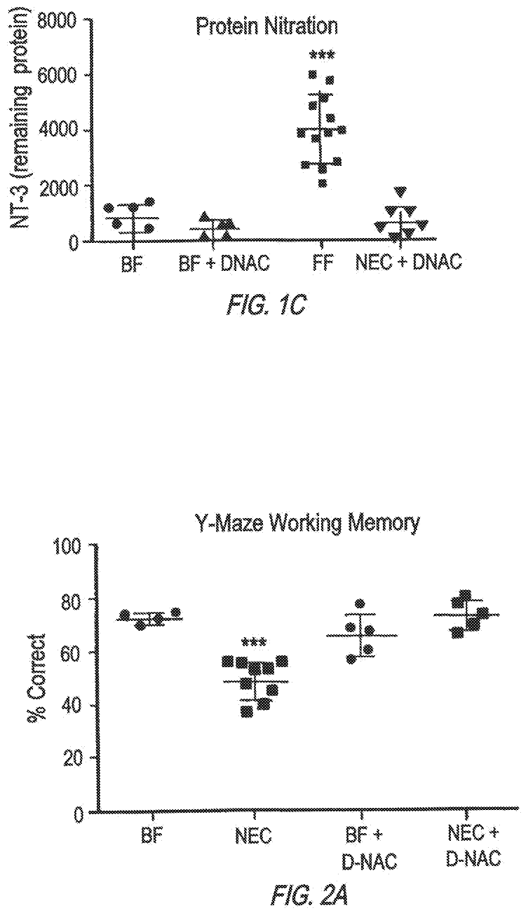

N-acetyl-L-cysteine (NAC) coupled dendrimers (D-NAC−100 mg/kg×2 doses) were delivered by oral administration 48 hours after the initiation of the experimental NEC protocol to both mice with NEC and age-matched breast-fed controls. This time point was selected in order to establish the effect of D-NAC on the brain while avoiding the potential effect that the dendrimers could have on the intestinal pathology. In addition, the selection of this time point was based on the observation that microglial activation is present as early as 48 hours after the initiation of the NEC protocol. The targeted delivery of D-NAC to the brain has been shown to be predominantly localized to the areas of greater microglial activation. In the experimental model, it was demonstrated that these nanoparticles localize predominantly to the periventricular and hippocampus area of the brain by using Cy5-labeled dendrimers (D-Cy5) administered orally 16 hours before the end of the experimental NEC protocol and evaluated in whole-brain sagittal sections of samples obtain from mice with NEC and compared to age-matched breast-fed controls (FIG. 1). Having demonstrated the specific localization of the dendrimers to the areas in which we see the most microglial activation, we evaluated the effects of D-NAC on the NEC-induced brain injury. Strikingly, it was found that oral administration of D-NAC 48 and 72 hours after the initiation of the NEC protocol led to a significant decrease in Iba-1 immunostaining as evaluated in whole-brain sagittal sections, indicating a decrease in microglial activation. The findings were particularly prominent in the periventricular area and the hippocampus. Furthermore, D-NAC administration 48 and 72 hours after the NEC protocol had been initiated was found to prevent the myelination impairment that had been observed in mice with NEC. As demonstrated by MBP immunohistochemical staining of whole-brain sagittal sections, NEC+D-NAC treated mice displayed an MBP expression profile that was not different than the age-matched breast-fed controls (FIG. 2), suggesting an adequate expression of the myelin components. Is important to highlight that D-NAC administration did not prevent the intestinal pathology, therefore the effect of D-NAC was exclusive to the NEC-induced brain injury.

Microglial activation has been demonstrated to lead to significant oxidative stress injury in the brain. Therefore, considering the findings of increased oxidative injury within the periventricular area of the brains of mice with NEC and the beneficial effects that delivery of NAC (an antioxidant) to the brain has on microglial activation (FIG. 2), and preservation of the normal myelination pattern (FIG. 2). It was not known whether or not NEC leads to oxidative injury in the brain. It was therefore determined if there was any evidence of accumulation of reactive oxygen species (ROS) within the brains of mice with NEC. Using the superoxide indicator dihydroethidium (DHE) in whole-brain sagittal sections, it was determined by immunofluorescent microscopy that the brains from mice with NEC are characterized by increased DHE uptake within the periventricular area and the hippocampus region, which suggested the presence of increased ROS production in these areas of the brain. Furthermore, it was evaluated whether or not D-NAC prevented the accumulation of ROS in mice with NEC. It was found that D-NAC administration 48 and 72 hours after the initiation of the NEC protocol, led to a significant decrease in the DHE uptake, indicating the absence of ROS accumulation.

To further determine the level of oxidative stress injury and to understand the mechanism(s) by which D-NAC may exert its neuro-protective effects, oxidative stress in the same region (i.e. the periventricular area and the hippocampus) was examined by quantifying markers of free radical injury to proteins and total levels of the major intracellular antioxidant glutathione (GSH). It was found that the periventricular region of the brain isolated from mice with NEC displays a significant increase in the oxidative injury to proteins by ONOO⁻ (peroxynitrite), as evidenced by the increased levels of 3-nitrotyrosine (NT-3, FIG. 2). Strikingly, administration of 2 doses of D-NAC orally prevented the oxidative injury to proteins as demonstrated by the decreased levels of NT-3 compared to mice with NEC (FIG. 2). NT-3 levels in the NEC+D-NAC mice were not different from the controls both with and without D-NAC, indicating that D-NAC prevents against oxidative injury to proteins. To further assess the effect of D-NAC on the brain, the total levels of GSH were quantified, which is critical in the brain to protect cells from free radical damage due to its potent antioxidant effects. The periventricular region of the brain of mice with NEC was analyzed and found a significant decrease in total glutathione (GSH) levels compared to breast-fed controls (FIG. 2); further demonstrating an oxidative process that led to the significant depletion of the antioxidant stores of the cell. On the other hand, total GSH levels in the periventricular region of mice with NEC that also received D-NAC were found to be similar to the breast-fed controls with and without D-NAC (FIG. 2), indicating that D-NAC prevents against the depletion of the antioxidant stores within the brain of mice with NEC. These findings indicate that NAC-coupled dendrimers unload NAC within the brain and enhance the cysteine pool available to maintain homeostatic concentrations of GSH, providing a critical buffer of protection for cellular milieu affected by NEC.

Having demonstrated the significant neuro-protective effect of D-NAC at the cellular and molecular level, it was determined whether or not D-NAC had an effect on the behavioral phenotype observed in mice with NEC, namely impaired cognitive performance (FIG. 2A-2D). In order to assess, the behavioral effects of D-NAC in mice with NEC, a modified version of the NEC was prepared. Briefly, animals were allowed to survive the NEC model after being exposed to a shorter version of our standard experimental protocol (3 days as opposed to 5 days). At the end of the 3 days of NEC, pups (age P10) were returned to the dam and allowed to recover until the time to perform behavioral testing. D-NAC (100 mg/kg) was administered orally as described above (i.e. 48 and 72 hours after the onset of the experimental protocol). Behavioral assessments (i.e. Y-maze spontaneous alternations, novel-object recognition and Morris water-maze test) were performed at weaning age (2-3 weeks after the induction of NEC, i. e. age P21). It was found that as opposed to the mice with NEC, mice that received D-NAC demonstrated no cognitive deficits in either of the tests performed (FIG. 2A-2D). In particular, NEC+D-NAC mice when compared to age-matched breast-fed controls displayed a similar percentage of correct spontaneous alternations in the Y-maze test (FIG. 2A), demonstrating no deficits in their working memory. A similar finding was evident in the case of the novel-object recognition test in which mice with NEC that received D-NAC were found to perform comparably to the age-matched breast fed controls (FIG. 2B), which demonstrated that D-NAC prevented the effects of NEC on recognition memory and retention ability. Evaluation of spatial memory and learning was performed using the Morris water maze, which evidenced as well that the cognitive function in mice with NEC that receive D-NAC was preserved and maintained at comparable levels to the age-matched breast-fed controls throughout the 1 week training period and at the time of the 48 hour post-training probe as described in the Methods section.

It was established whether or not the effect of NEC on the brain and the preventive properties of D-NAC were restricted to the acute and early-stages of the disease process. The myelination profile in mice submitted to the experimental NEC protocol as well as those that received D-NAC 60 days after the experimental protocol had been concluded (age P70) were compared. A significant decrease in microglial activation by Iba-1 staining (FIG. 1A) was seen in the periventricular region in NEC pups treated with D-NAC, by IHC and confirmed by quantification, that is similar to healthy controls. This is associated with increased glutathione levels and decreased protein nitration in pups with NEC that are treated with D-NAC (FIGS. 1B and 1C). D-NAC treatment in NEC pups was also associated with improved myelination when compared to untreated animals and the myelination was similar to that of healthy, breast fed controls. Similarly a decrease in oxidative stress markers DHE was seen in pups with NEC that were treated with D-NAC, compared to untreated NEC pups, and was similar to that seen in healthy breast fed controls. MBP expression evaluated in whole-brain sagittal sections by IHC and quantified (FIG. 1A) was found to be decreased even at this later age in mice submitted to the NEC protocol. In contrast, mice with NEC that received D-NAC displayed comparable levels of MPB expression as the age-matched breast-fed controls, demonstrating that the effects of NEC and the protective effect of D-NAC is not exclusive to the acute and early-stage of the disease process.

Taken together the findings demonstrate that oral administration of a dendrimer-coupled antioxidant (N-acetyl-cysteine—NAC) to mice with NEC prevents the onset of oxidative damage to the brain, subsequently preventing the myelination impairment and the cognitive deficit that was found to be characteristic of murine NEC, yet without an effect on the development of the intestinal disease.

Despite the fact that the experimental model includes a component of brief intermittent hypoxia (10 min twice a day for 4 days), the findings are not related to this component of the experimental protocol, as it was demonstrated that hypoxia alone as applied in the NEC protocol does not lead to impaired myelination. It is still unclear whether dysmyelination is due to i) direct loss of oligodendrocyte precursor cells (OPCs), which give rise to oligodendrocytes (OLs—the myelinating cells of the central nervous system—CNS); ii) disruption of the differentiation/developmental program of OPCs or iii) a combination of both effects in which impaired regeneration and repair of the initial loss of OPCs leads to rapid proliferation of premyelinating oligodendrocytes (preOLs) that subsequently fail to progress and mature to myelinating cells, ultimately leading to impaired white matter growth. As demonstrated in FIGS. 2A-2D, NEC leads to increased loss of OPCs by apoptosis, which was evident by the increased number of OPCs (Olig2+ cells) that are also TUNEL+. To further evaluate the effect of NEC on OPC viability and differentiation/maturation towards becoming myelinating OLs, a mouse strain that expresses membrane green fluorescent protein (GFP) under the control of the OPC lineage marker protein platelet derived growth factor receptor α (Pdgfrα—a well-established OPC cellular marker) was generated. Upon exposure of this mouse strain to the experimental model, it was determined that NEC leads to loss of OPC lineage cells (demonstrated by the decreased number of GFP+ cells) and to a significant impairment in the differentiation of preOLs towards myelinating OLs (evidenced by the decreased MBP expression).

Myelin basic protein mRNA expression was significantly decreased at P9 in mice with NEC. It is remarkable that when myelination was evaluated in the young adult mice that were allowed to survive a milder version of the experimental NEC model (3 days as opposed to 4 days), expression of myelin basic protein was found to be decreased even 60 days post injury (P70) as shown in FIG. 2E. These findings demonstrated that NEC-induced dysmyelination is a long-lasting effect that remains into young adulthood in rodents, which correlates with what has been demonstrated in school-aged human NEC survivors. It is of great importance to highlight the fact that the first and second postnatal weeks in rodents, which correspond to the last 10 weeks of human gestation, are the most critical in the normal differentiation and maturation of OPCs to OLs and therefore essential for optimal myelination of the brain. Therefore, during this developmental window, which coincides with the timing of the experimental model and the developmental stage of premature children afflicted with NEC, the premature brain is particularly susceptible to white matter injury (WMI). This particular type of injury has been clearly associated with neurodevelopmental impairment in children that survive NEC, clinical findings that are substantiated in the experimental model both at the behavioral as well as the anatomopathological level.

### EXAMPLE 2

### Testing in Piglet Model

Similar results have been obtained using a piglet model, described by Good, et al. Am J Physiol Gastrointest Liver Physiol. 2014 306(11):G1021-32. doi: 10.1152/ajpgi.00452.2013. Epub 2014 Apr. 17.

### EXAMPLE 3

### Corroboration of Mouse Results in Human Brain From Infants Who Died From NEC

The findings in mice were corroborated in studies of human brain obtained from infants who died from NEC, and were not seen in age matched control brains. Mouse and human brains with NEC revealed increased apoptosis and impaired differentiation of myelin producing oligodendrocyte progenitor cells (OPCs) and activation of the immune modulating microglial cells in the corpus callosum, hippocampus and midbrain, leading to the release of reactive oxygen species (ROS) and evidence of oxidative injury

All references, including publications, patent applications, and patents, cited herein are hereby incorporated by reference to the same extent as if each reference were individually and specifically indicated to be incorporated by reference and were set forth in its entirety herein.

1-18. (canceled)

19. A method of treating one or more symptoms of a gastrointestinal disorder in a subject, the method comprising:
administering to the subject a composition comprising a poly(amidoamine) (PAMAM) hydroxyl-terminated dendrimer conjugated to one or more anti-inflammatory agents,
wherein the gastrointestinal disorder is associated with inflammation in the gastrointestinal tract and/or central nervous system of the subject.

20. The method of claim 19, wherein the dendrimer is not conjugated to a targeting moiety.

21. The method of claim 19, wherein the dendrimer is a G3, G4, G5, or G6 PAMAM dendrimer.

22. The method of claim 19, wherein the dendrimer is conjugated to each of the one or more anti-inflammatory agents via a disulfide bond.

23. The method of claim 19, wherein the dendrimer is conjugated to each of the one or more anti-inflammatory agents via a spacer compound.

24. The method of claim 23, wherein the spacer compound is selected from the group consisting of N-succinimidyl 3-(2-pyridyldithio)propionate (SPDP), Glutathione (GSH), Gamma-aminobutyric acid (GAB A), and combinations thereof.

25. The method of claim 19, wherein the gastrointestinal disorder is necrotizing enterocolitis (NEC).

26. The method of claim 25, wherein the gastrointestinal disorder is NEC, and the dendrimer is in an amount effective to alleviate one or more symptoms of necrotizing enterocolitis in the subject.

27. The method of claim 19, wherein the gastrointestinal disorder is associated with central nervous system complications.

28. The method of claim 19, wherein the anti-inflammatory agent is N-acetyl cysteine.

29. The method of claim 19, wherein the anti-inflammatory agent is an inhibitor of toll like receptor 4.

30. The method of claim 29, wherein the inhibitor of toll like receptor 4 agent is C34, a 2-acetamidopyranoside with the formula C₁₇H₂₇NO₉, or salt thereof.

31. The method of claim 19, wherein the composition further comprises a dendrimer conjugated to an anti-excitotoxic agent or an anti-glutamate agent.

32. The method of claim 19, wherein the composition is formulated in a suspension, emulsion, tablet, capsule, or lavage.

33. The method of claim 19, wherein the composition is formulated in a formula for infants.

34. A composition for use according to the method of claim 19.
